(19) United States
(12) Patent Application Publication
(10) Pub. No.: US 2010/0262024 A1
(43) Pub. Date: Oct. 14, 2010

(54) HEART RATE VARIABILITY MEASUREMENT METHOD

(75) Inventors: WEI-CHIH HU, CHUNG LI (TW); CHAO-FENG CHANG, CHUNG LI (TW)

Correspondence Address: ROSENBERG, KLEIN & LEE, 3458 ELLICOTT CENTER DRIVE-SUITE 101, ELLICOTT CITY, MD, 21043, US

(73) Assignee: CHUNG YUAN CHRISTIAN UNIVERSITY, CHUNG LI (TW)

(21) Appl. No.: 12/726,827

(22) Filed: Mar. 18, 2010

(30) Foreign Application Priority Data
Apr. 9, 2009 (TW) 098111870

(51) Int. Cl. A61B 5/02

(52) U.S. Cl. 600/504

(57) ABSTRACT

A heart rate variability measurement method is revealed. The method includes the following steps. At first, play a piece of music for a participant. Then detect a change in vascular volume of the participant. Next depict a continuous waveform signal of the change in vascular volume. Thus while measuring the heart rate as well as heart rate variability, the participant is not easy to get nervous or impatient and the real heart rate as well as heart rate variability can be obtained, without being affected by the mood. Therefore, the accuracy of the heart rate variability during measurement is increased.

### BACKGROUND OF THE INVENTION

1. Field of Invention

The present invention relates to a measurement method, especially to a heart rate variability measurement method.

2. Description of Related Art

The heart rate in the Heart Rate Variability (HRV) is a measure of the number of heart beats and its unit is beat per minute (bpm). In 1981, Akselrod reported that electrocardiography is measured and recorded by a non-invasive way. By using Fast Fourier Transforms (FFT), heart rate variability power spectra were obtained. The variability represents the difference between the beat-to-beat intervals. That means the variation of beat-to-beat intervals and the amount of variation at specific frequencies. The heart rate variability power spectra correspond to the physiological activity of the autonomic nervous system. Long term HRV reflects disorders of the autonomic nervous system, overall health of hearts and cardiac functions.

The autonomic nervous system (ANS or visceral nervous system) is the part of the peripheral nervous system that controls functions of organs inside the body. It can be divided by subsystems into the parasympathetic nervous system and sympathetic nervous system. The sympathetic nervous system is always active at a basal level and becomes more active during times of stress. The sympathetic nervous system is called into action and it uses energy. These actions help us to fight against during emergent situations. As to the parasympathetic nervous system, it becomes active during the rest and relaxed state. The actions of the parasympathetic nervous system increases digestion, recuperation and circulation. In order to let our bodies have a good rest, the parasympathetic nervous system promotes energy conservation.

The most effective method that evaluates activity of the autonomic nervous system available now is HRV analysis. The HRV changes through the interaction and regulation of the sympathetic nervous system and parasympathetic nervous system. In medical science, the neural regulation of the autonomic nervous system is studied by the HRV. From alternation of HRV, whether a person suffers from malfunction of the automatic nervous system can be found. Moreover, HRV also reflects heart functions. Low HRV indicates high cardiac risk. Thus alteration of HRV has been reported to be associated with various pathologic conditions or therapies such as cardiac arrhythmias, diabetes, depression, and so on.

The cardiac arrhythmia means irregular heart beat, variations in beat-to-beat intervals. The heart beat may be too fast or too slow. For example, tachycardia refers to a heart rate that exceeds the normal range for a resting heart rate while Bradycardia is defined as a resting heart rate under the normal heart beats. Besides some cardiac diseases, the breathing status is also associated with cardiac arrhythmia. While breathing in deeply, the heart beat quickens but while breathing out, the heart beat slows. Moreover, the heart beat increases if someone takes exercise while the heart beat slows down at rest or sleeping. Furthermore, coffee, tea, fever, tension, stress, pain, anoxia and drugs etc. that causes arousal of the automatic nervous system all may change the heart beat rate and rhythm.

Some arrhythmias may have no symptoms at all while others have some minor ones such as rapid heartbeat or feeling of irregular heartbeat. Serious arrhythmias symptoms cause patients shock, syncope and sudden death. Some patients of sudden death have no symptoms at all and young people may also have sudden death. In the medical field, sudden decrease of HRV can be used as a predictive indicator of diseases besides cases analysis. Especially to the people who are always busy, their HRV should be monitored in a long term. Once the HRV is reduced or decreased gradually, they will take a rest so as to reduce the risk of sudden death.

Moreover, HRV can be used to evaluate therapeutic effects of diabetes patients. In early stage of diabetes, HRV has already decreased gradually although the blood sugar is within the normal range. In the middle and late stages, the patients may have complications such as diabetic neuropathy and small nerves of sympathetic and parasympathetic nervous systems start to have necrosis. Patients have symptoms of disorders of the autonomic nervous system (ANS) such as posture vertigo (low blood pressure), palpitation, night sweat, diarrhea and so on. By long term monitoring of HRV, it is found that the HRV curve diverges from the baseline. The therapeutic effects can also be evaluated in this way.

Furthermore, HRV can also check the onset of depression. Depression is not only a depressed mood. Over ten million people suffer from the illness each year and the risk of depression among females are twice that of males. Patients with other diseases such heart diseases, stroke, cancers and diabetes are at higher risk of getting depression. These patients often have lower activity measurements of HRV. The articles show that many prescription drugs relieve symptoms of depression. According to statistics, about 80% to 90% depression patients can be cured by drugs and psychotherapy. Once HRV is applied to evaluate therapeutic effects in a long run, the depression is relieved more quickly.

Without detailed analysis of the whole ECC, the HRV data can be obtained, only by the beat-to-beat interval. It takes a period of time, about 10 minutes, to measure HRV. Then through the re-sampling process, the sampled data is processed by Fast Fourier Transforms (FFT) so as to get heart rate variability power spectrum. High frequency (0.15-0.4 Hz) and low frequency (0.04-0.15 Hz) spectra power can be obtained from the heart rate variability power spectrum. The change of high and low frequency power is used as an index of activity of the autonomic nervous system.

However, during the long-term measurement, once the participants' attention focuses on this event (test), they may feel nervous or impatient and their physiological conditions are also affects. Thus the real physiological data is unable to get. Some conditions or diseases are unable to be detected with short-term measurement and nervous, impatient participants. For example, sporadic arrhythmia can only be observed under the long-term measurement.

Thus there is a need to provide a heart rate measurement method that won't make participants feel nervous or annoying so as to detect the heart rate variability of the participant and obtain heart rate and heart rate variability in a relaxed state, without the influence of nervous mood.

### SUMMARY OF THE INVENTION

Therefore it is a primary object of the present invention to provide a heart rate variability (HRV) measurement method in which a change in vascular volume of a participant after the participant listening to a piece of music being played. Thus the participant will not feel nervous or impatient so as to get heart rate and heart rate variability of the participant in a relaxed state. Therefore, the accuracy of the HRV measurement is increased, without being affected by the mood of the participant.

In order to achieve above object, a heart rate variability (HRV) measurement method of the present invention includes the following steps. At first, play a piece of music for a participant. Then measure a change in vascular volume of the participant. Next depict a continuous waveform signal of the change in vascular volume. Thus while measuring the heart rate as well as heart rate variability, the participant is not easy to get nervous or impatient and the real heart rate as well as heart rate variability can be obtained. Therefore, without being affected by the mood, the accuracy of the heart rate variability during measurement is increased.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a flow chart of an embodiment according to the present invention;

FIG. 2 is a flow chart of an embodiment showing measurement of changes in vascular volume of a participant according to the present invention;

FIG. 3 is a schematic drawing showing structure of reflectance measurement of an embodiment according to the present invention;

FIG. 4 is a flow chart of an embodiment showing how sensing signals are generated according to the present invention;

FIG. 5 is a flow chart of another embodiment showing measurement of changes in vascular volume of a participant according to the present invention;

FIG. 6 is a schematic drawing showing structure of pass-through type measurement of an embodiment according to the present invention;

FIG. 7 is a flow chart of another embodiment showing how sensing signals are generated according to the present invention.

### DETAILED DESCRIPTION OF THE PREFERRED EMBODIMENT

When someone feels anxious or irritated, obvious irregular waves appear in his HRV spectrum. Once he is in a relaxed and peaceful state, the HRV spectrum shows regular peaks. Thus the HRV provides valuable insight into people's physiological conditions and also works as an indicator for making diagnoses and choice of therapies. Therefore, while detecting the heat rate and its variability, the participants should feel relaxed so as to get real data.

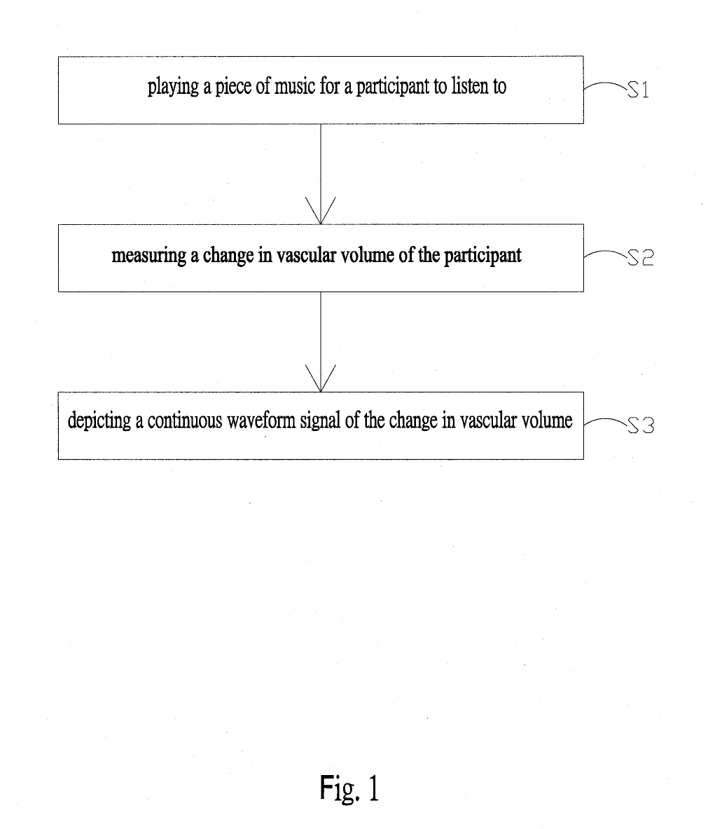

Refer to FIG. 1, a flow chart of an embodiment according to the present invention is revealed. As shown in the figure, heart rate variability measurement method includes the following steps. At first, take the step S1, play a piece of music for a participant. Then take the step S2, detect a change in vascular volume of the participant. Next, run the step S3, a continuous waveform signal corresponding to the change in vascular volume is drawn.

Music has physiological and psychological effects on people. As to the physiological effect, music activates the autonomic nervous system and acts in regulation of heart beat, respiratory rate, nerve conduction, blood pressure and internal secretions. The psychological effect of the music includes induction of autonomic response in the cerebrum that controls human emotions and feelings so that the mood changes. People's respiration and heat beat represent natural rhythms of the bodies. By physiological and psychological effects of the music, the body rhythms can be changed so as to achieve a harmony between the body rhythms and the music rhythm.

The psychosomatic effects caused by music mainly come from limbic system and the hypothalamus in the brain affected by melodies and rhythms of the music. Then the endocrine system and the autonomic nervous system are further regulated. The physiological conditions are affected by music rhythm more than the preference of music. The music with fast tempos leads to increased blood pressure, higher heart rate, and increased ratio of low-to-high frequency spectra power (LF/HF). The sympathetic nervous system is activated. While listening to music with slow tempos or meditation music, people feel relaxed. Thus before measurement of HRV, the participant listen to soft and gentle music by a speaker or an earphone so as to be in a relaxed mood. Thus the participant will not become nervous or impatient while measuring the heart rate and HRV. Therefore, the real heart rate and HRV values are obtained, without being affected by the nervous mood and the accuracy of the measurement is increased.

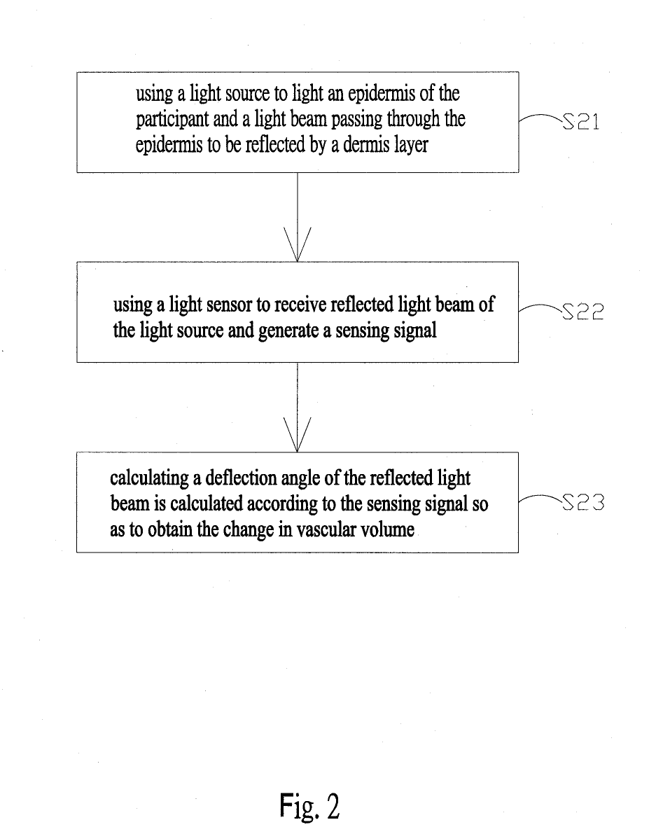

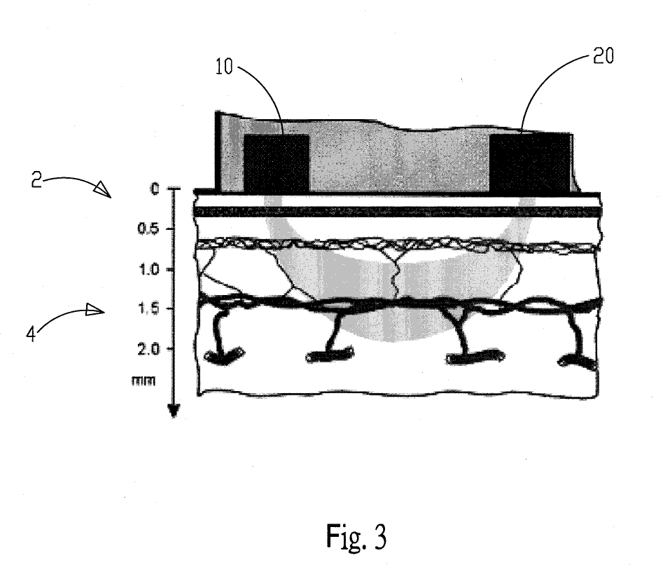

Refer to FIG. 2 & FIG. 3, a flow chart of measurement of the change of vascular volume of the participant as well as a schematic drawing of the reflectance measurement is revealed. The measurement of a change in vascular volume of the participant consists of the following steps: firstly, take the step S21, a light source 10 is provided to light an epidermis 2 of the participant. A light beam passes through the epidermis 2 to be reflected by a dermis layer 4. Then take the step S22, use a light sensor 20 to receive reflected light beam of the light source 10 and generate a sensing signal. The light sensor 20 is a phototransistor. Next, run the step S23 according to the sensing signal, a deflection angle of the reflected light beam is calculated and the change in vascular volume is obtained.

In the present invention, photoplethysmograph (PPG) is used to record physiological signals. In this method, a LED (light emitting diode) is used as a light source 10 and a phototransistor is used as a main measurement device of PPG. Photoplethysmograph is based on measurement of changes in light absorption of a selected skin area. Generally, a near-infrared light source 10 is used to illuminate the skin. When light passes the biological tissue, it is absorbed by various materials such as skin pigments, bones, arteries, and veins. Moreover, the artery blood vessel contains more blood during systole (contraction) than diastole (relaxation). The arterial diameter also increases due to increasing of the pressure. These effects only occur in arteries and small arteries, not the veins.

During heart muscle contraction, the light absorption increases and this is due to large amount of materials (such as hemoglobin) that absorbs light entering the blood and the increasing travel distance of the light in the artery. As to the total absorption, the light absorbing materials are like alternating components. The alternating components help us to differentiate light absorption of unfluctuating components such as materials with certain amount in arterial or venous blood, skin pigments and so on (direct component) and light absorption of fluctuant components in the arteries (alternating component). The alternating component is only 1%-2% of direct component. Thus optical signal waveforms changing along with time and tissues are called photoplethysmograph (PPG). By the PPG technique, the fluctuation of arteries is reflected by the absorption of light so as to analyze cardiovascular parameters.

The flow of blood in the vessels is particularly affected by heartbeat so as to have periodic changes. And the blood pressure is further influenced by such changes. Continuous changes of pressure in the flexible blood vessels lead to changes of diameters of blood vessels. Such changes of diameters of blood vessels are also continuous due to continuous changes of blood pressure. In order to measure such physical quantity, the optical inspection is used. As described in the above embodiment, light beams emitted from the light source 10 is reflected by the dermis layer 4 and finally arriving the light sensor 20. The changes of diameters of blood vessels cause deflection of light. Then continuous waveform signals of blood vessels are calculated and obtained by these deflection angles.

Generally, fiber optic sensors are applied to measure PPG and the spectrum analysis of the signals is done. The result shows that power spectra (the distribution of power) of PPG and ECG are consistent. Moreover, from previous articles, it was found that heart rate and respiratory rate can be obtained from PPG signals by digital filtering techniques. The correlation between the heart rate from PPG signals and the heart rate from ECG signals is 0.99. Thus among normal people, no matter in view of the time domain or the frequency domain, the peak-interval of PPG and that of ECG are similar to each other. Thus HRV data obtained by analyses of these two peak-intervals show no significant difference. Therefore, by continuous waveform signals of the changes in vascular volume depicted by PPG, a corresponding power spectrum of ECG is obtained.

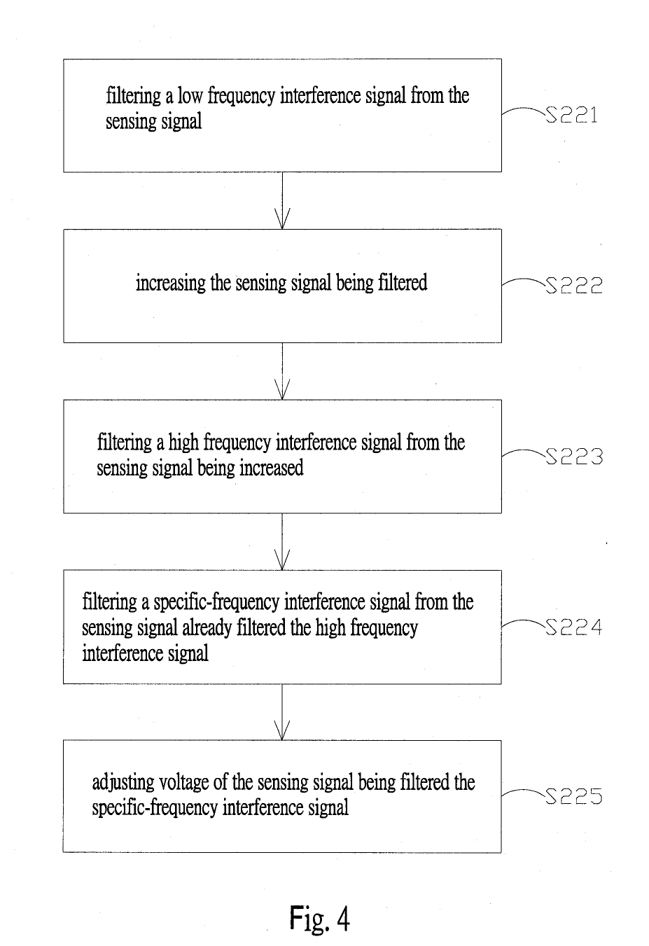

Refer to FIG. 4, a schematic drawing of an embodiment according to the present invention showing how sensing signals are generated. As shown in figure, after the step S22, the following steps are run. At first, take step S221, filter a low frequency interference signal from the sensing signals. Low frequency drift components are removed by a high pass filter so that low frequency interference during measurement can be avoided. Then take the step S222, increase the sensing signals being filtered. The signals are amplified ten times by means of a reverse amplification circuit. Next, take the step S223, filter a high frequency interference signal from the sensing signals being increased. A low pass filtering effect is achieved by a low pass filter so as to avoid high frequency interference during measurement of changes in vascular volume. Because the frequency of most PPG signals falls within 10 Hz, 60 Hz household appliance noise can be filtered by the low pass filter. And the cutoff frequency is set at 10 Hz, working as a pilot filter that filters all 60 Hz signals at once. Later take the step S224, filter a specific-frequency interference signal from the sensing signals already filtered the high frequency interference signal. Use a band-reject filter to block signals within a band of frequencies, especially for filtering signals at specific frequency from unknown signals. In this embodiment, 60 Hz power supply noise is filtered. At last, run the step S225, adjust the voltage of the sensing signals being filtered the specific-frequency interference signal. The direct current (DC) level of the signals is adjusted by an amplifier and a subtract amplifier and the signals are controlled within a specific voltage range. Thus while depicting continuous waveform signals, the continuous changes in waveform signals obtained are more accurate so as to take the following step S23-calculate deflection angles of the reflected light according to the sensing signals for the changes in vascular volume.

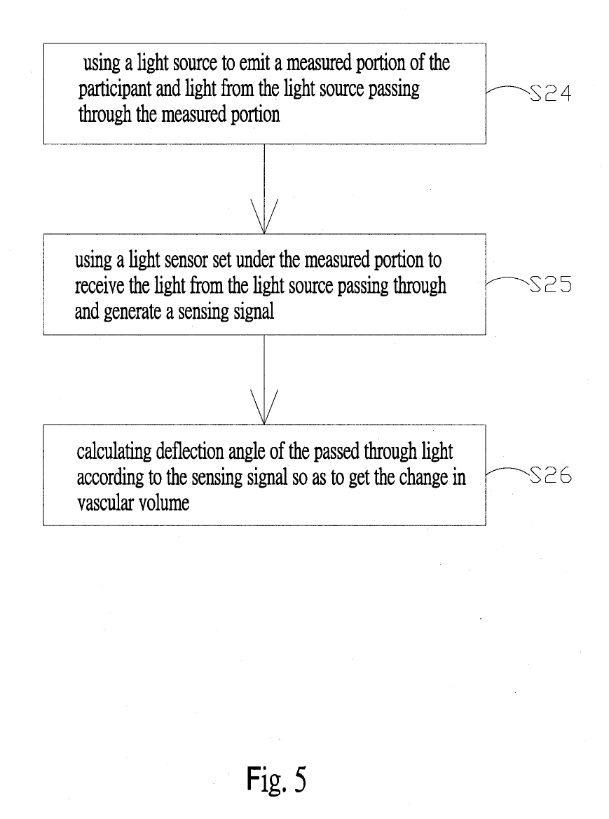

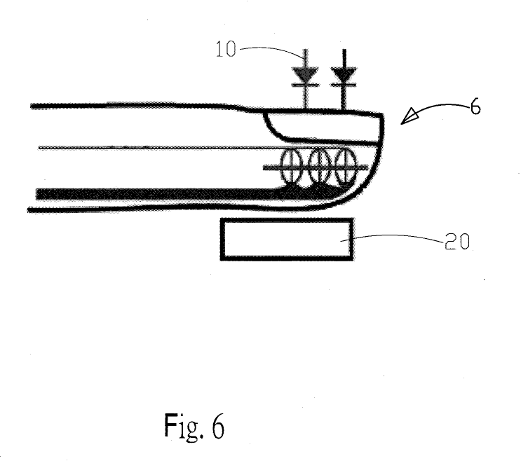

Refer to FIG. 5 and FIG. 6, a flow chart showing measurement of changes in vascular volume of participants of another embodiment and a pass-through type measurement structure are revealed. As shown in figure, while measuring the changes in vascular volume of participants, firstly take the step S24, use a light source 10 emits a measured portion 6 of the participant. Then run the step S25, a light sensor 20 is set under the measured portion 6 to receive the light passing through and generate a sensing signal. The light sensor 20 is a phototransistor. Next take the step S26, calculate the deflection angle of the passed through light according to the sensing signal to get the change in vascular volume. The difference between this embodiment and above one is in that the measurement of the above one is based on reflection while the measurement of this embodiment is a pass-through type. Thus the light from the light source 10 penetrates the tissues of the measured portion 6 to be received by the light sensor 20 thereunder.

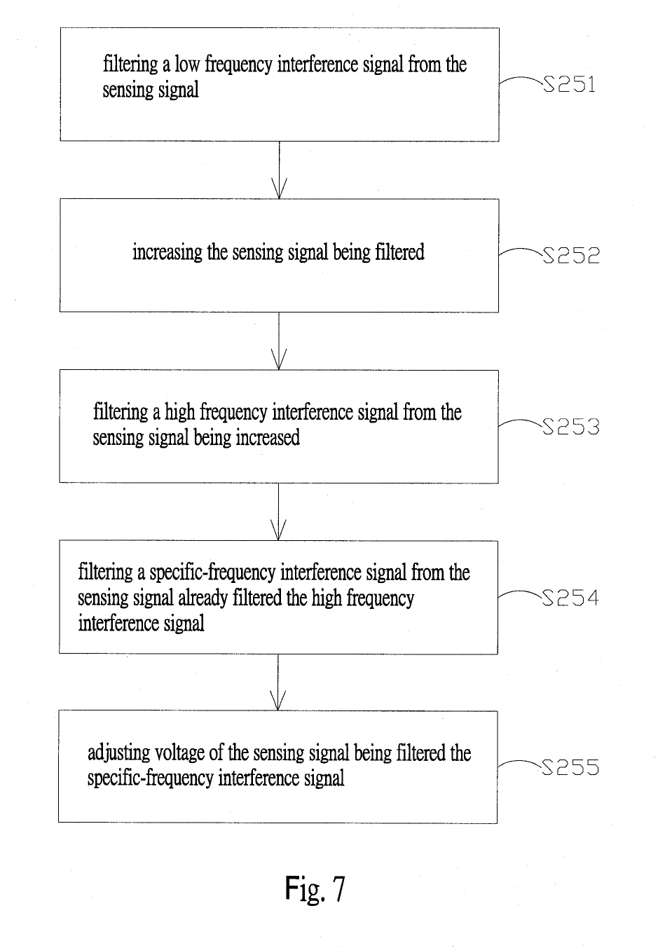

Refer to FIG. 7, a further embodiment of the present invention showing steps of generating sensing signals is revealed. As shown in figure, after the step S25, the following steps are taken. In the beginning, take the step S251, filter a low frequency interference signal from the sensing signals. Then take the step S252, increase the sensing signals being filtered. Next, take the step S253, filter a high frequency interference signal from the sensing signals being increased. Later take the step S254, filter a specific-frequency interference signal from the sensing signals already filtered the high frequency interference signal. At last, run the step S255, adjust the voltage of the sensing signals being filtered the specific-frequency interference signal. Thus the continuous changes in waveform signals obtained are more accurate while depicting continuous waveform signals and the following step S25 can be run-calculate deflection angles of the reflected light according to the sensing signals to get the changes in vascular volume.

Refer to the list 1, list 2 and list 3. The list one shows low and high frequency components of 20 participants under the environment without listening to music measured by PPG and after normalization. The list two shows low and high frequency components of 20 participants under the environment listening to music measured by PPG and after normalization. The list 3 shows LF (low frequency)/HF (high frequency) ratio of 20 participants under the environment with and without listening to music measured by PPG and after normalization. The HRV parameters mainly include low frequency components, high frequency components, and LF/HF ratio. The low frequency components can be used as a quantitative index of the sympathetic nervous activity while the sympathetic nervous system dominates in stressful situations. The high frequency components can be used as a quantitative index of the parasympathetic nervous activity and the parasympathetic nervous system dominates during rest or relaxed state. As to the LF/HF ratio, it is used as an indicator of balance between sympathetic and parasympathetic nervous systems.

List 1

| | Without listening | Without listening |
|---|---|---|
| | to music PPG(LF) | to music PPG(HF) |
| Subject1 | 0.469 ± 0.073 | 0.53 ± 0.073 |
| Subject2 | 0.65 ± 0.056 | 0.349 ± 0.056 |
| Subject3 | 0.547 ± 0.07 | 0.452 ± 0.07 |
| Subject4 | 0.496 ± 0.069 | 0.503 ± 0.069 |
| Subject5 | 0.519 ± 0.052 | 0.48 ± 0.052 |
| Subject6 | 0.557 ± 0.065 | 0.442 ± 0.065 |
| Subject7 | 0.517 ± 0.078 | 0.482 ± 0.078 |
| Subject8 | 0.556 ± 0.075 | 0.443 ± 0.075 |
| Subject9 | 0.522 ± 0.042 | 0.478 ± 0.042 |
| Subject10 | 0.57 ± 0.061 | 0.429 ± 0.061 |
| Subject11 | 0.65 ± 0.052 | 0.35 ± 0.052 |
| Subject12 | 0.56 ± 0.057 | 0.44 ± 0.057 |
| Subject13 | 0.555 ± 0.086 | 0.444 ± 0.086 |
| Subject14 | 0.469 ± 0.079 | 0.53 ± 0.079 |
| Subject15 | 0.561 ± 0.064 | 0.439 ± 0.064 |
| Subject16 | 0.526 ± 0.069 | 0.473 ± 0.069 |
| Subject17 | 0.508 ± 0.062 | 0.492 ± 0.062 |
| Subject18 | 0.477 ± 0.056 | 0.522 ± 0.056 |
| Subject19 | 0.512 ± 0.087 | 0.487 ± 0.087 |
| Subject20 | 0.444 ± 0.084 | 0.555 ± 0.084 |

List 2

| | Listening to | Listening to |
|---|---|---|
| | music PPG(LF) | music PPG(HF) |
| Subject1 | 0.42 ± 0.068 | 0.579 ± 0.068 |
| Subject2 | 0.493 ± 0.062 | 0.506 ± 0.062 |
| Subject3 | 0.534 ± 0.054 | 0.465 ± 0.054 |
| Subject4 | 0.38 ± 0.065 | 0.619 ± 0.065 |
| Subject5 | 0.482 ± 0.041 | 0.517 ± 0.041 |
| Subject6 | 0.451 ± 0.088 | 0.548 ± 0.088 |
| Subject7 | 0.49 ± 0.066 | 0.509 ± 0.066 |
| Subject8 | 0.524 ± 0.068 | 0.475 ± 0.068 |
| Subject9 | 0.443 ± 0.09 | 0.556 ± 0.09 |
| Subject10 | 0.394 ± 0.07 | 0.605 ± 0.07 |
| Subject11 | 0.559 ± 0.088 | 0.441 ± 0.088 |
| Subject12 | 0.537 ± 0.05 | 0.462 ± 0.05 |
| Subject13 | 0.472 ± 0.061 | 0.527 ± 0.061 |
| Subject14 | 0.341 ± 0.059 | 0.658 ± 0.059 |
| Subject15 | 0.517 ± 0.08 | 0.482 ± 0.08 |
| Subject16 | 0.505 ± 0.056 | 0.495 ± 0.056 |
| Subject17 | 0.442 ± 0.058 | 0.557 ± 0.058 |
| Subject18 | 0.386 ± 0.043 | 0.613 ± 0.043 |
| Subject19 | 0.42 ± 0.085 | 0.58 ± 0.085 |
| Subject20 | 0.437 ± 0.052 | 0.562 ± 0.052 |

List 3

| | Without listening | Listening to |
|---|---|---|
| | to music PPG(LF/HF) | music PPG(LF/HF) |
| Subject1 | 0.923 ± 0.27 | 0.751 ± 0.21 |
| Subject2 | 1.942 ± 0.54 | 1.005 ± 0.24 |
| Subject3 | 1.266 ± 0.36 | 1.187 ± 0.24 |
| Subject4 | 1.028 ± 0.31 | 0.632 ± 0.16 |
| Subject5 | 1.105 ± 0.24 | 0.942 ± 0.15 |
| Subject6 | 1.307 ± 0.34 | 0.875 ± 0.34 |
| Subject7 | 1.125 ± 0.32 | 0.995 ± 0.24 |
| Subject8 | 1.319 ± 0.38 | 1.144 ± 0.3 |
| Subject9 | 1.109 ± 0.19 | 0.847 ± 0.32 |
| Subject10 | 1.379 ± 0.37 | 0.675 ± 0.2 |
| Subject11 | 1.924 ± 0.44 | 1.345 ± 0.41 |
| Subject12 | 1.31 ± 0.29 | 1.201 ± 0.29 |
| Subject13 | 1.342 ± 0.48 | 0.921 ± 0.22 |
| Subject14 | 0.927 ± 0.29 | 0.53 ± 0.14 |
| Subject15 | 1.323 ± 0.33 | 1.134 ± 0.4 |
| Subject16 | 1.156 ± 0.31 | 1.042 ± 0.23 |
| Subject17 | 1.066 ± 0.27 | 0.812 ± 0.18 |
| Subject18 | 0.936 ± 0.22 | 0.639 ± 0.12 |
| Subject19 | 1.115 ± 0.37 | 0.772 ± 0.34 |
| Subject20 | 0.845 ± 0.3 | 0.792 ± 0.16 |

It is learned from above lists, the high frequency spectra of participants is dramatically raised under the environment with music. That represents the participants are more relaxed. Thus the participants will not feel nervous or impatient.

In summary, a heart rate variability measurement method of the present invention includes the following steps. At first, play a piece of music for a participant. Then detect a change in vascular volume of the participant. Next depict a continuous waveform signal of the change in vascular volume. Thus the participant is not easy to get nervous or impatient and the real heart rate as well as heart rate variability of the participant can be measured and obtained. Therefore, the accuracy of the heart rate variability during measurement is increased.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A heart rate variability measurement method comprising the steps of:
playing a piece of music for a participant to listen to,
measuring a change in vascular volume of the participant, and
depicting a continuous waveform signal of the change in vascular volume.

2. The method as claimed in claim 1, wherein the step of measuring a change in vascular volume of the participant includes the steps of:
using a light source to light an epidermis of the participant and a light beam passing through the epidermis to be reflected by a dermis layer,
using a light sensor to receive reflected light beam of the light source and generate a sensing signal, and
calculating a deflection angle of the reflected light beam is calculated according to the sensing signal so as to obtain the change in vascular volume.

3. The method as claimed in claim 2, wherein, after the step of generating a sensing signal, the method further includes the steps of:
filtering a low frequency interference signal from the sensing signal,
increasing the sensing signal being filtered,
filtering a high frequency interference signal from the sensing signal being increased,
filtering a specific-frequency interference signal from the sensing signal already filtered the high frequency interference signal, and
adjusting voltage of the sensing signal being filtered the specific-frequency interference signal.

4. The method as claimed in claim 2, wherein in the step of using a light sensor to receive reflected light beam of the light source, a phototransistor is used to receive reflected light beam of the light source.

5. The method as claimed in claim 1, wherein the step of measuring a change in vascular volume of the participant includes the steps of:
using a light source to emit a measured portion of the participant and light from the light source passing through the measured portion,
using a light sensor set under the measured portion to receive the light from the light source passing through and generate a sensing signal, and
calculating deflection angle of the passed through light according to the sensing signal so as to get the change in vascular volume.

6. The method as claimed in claim 5, wherein after the step of generating a sensing signal, the method further includes the steps of:
filtering a low frequency interference signal from the sensing signal, increasing the sensing signal being filtered,
filtering a high frequency interference signal from the sensing signal being increased,
filtering a specific-frequency interference signal from the sensing signal already filtered the high frequency interference signal, and
adjusting voltage of the sensing signal being filtered the specific-frequency interference signal.

7. The method as claimed in claim 5, wherein in the step of using a light sensor to receive reflected light beam of the light source, a phototransistor is used to receive reflected light beam of the light source.

8. The method as claimed in claim 1, wherein in the step of playing a piece of music for a participant to listen to, a speaker is used to play a piece of music for a participant to listening to.

9. The method as claimed in claim 1, wherein in the step of playing a piece of music for a participant to listen to, an earphone is used to play a piece of music for a participant to listen to.
